SEQUENCE LISTING (2 sequences)

SEQ ID NO: 1 - DNA, 21 bp, synthetic construct
  source 1..21: mol_type = other DNA
ggaacccctagtgatggagtt

SEQ ID NO: 2 - DNA, 16 bp, synthetic construct
  source 1..16: mol_type = other DNA
cggcctcagtgagcga

(19) United States
(12) Patent Application Publication
(10) Pub. No.: US 2023/0323397 A1
(43) Pub. Date: Oct. 12, 2023

(54) WATER-BASED HYPOTONIC LYSIS METHOD FOR ADENO-ASSOCIATED VIRUS PURIFICATION

(71) Applicant: UNIVERSITY OF SOUTH CAROLINA, COLUMBIA, SC (US)

(72) Inventors: SUJIT PUJHARI, COLUMBIA, SC (US); ROBERT ANDERSON SMITHWICK, GREENVILLE, SC (US)

(21) Appl. No.: 18/100,028

(22) Filed: Jan. 23, 2023

Related U.S. Application Data
(60) Provisional application No. 63/398,622, filed on Aug. 17, 2022.
(60) Provisional application No. 63/322,796, filed on Mar. 23, 2022.

(51) Int. Cl. C12N 15/113; C12N 9/22; C12N 15/86

(52) U.S. Cl. CPC C12N 15/86; C12N 9/22; C12N 15/1131; C12N 2310/122; C12N 2750/14143

(57) ABSTRACT

Disclosed is a method of purifying recombinant adeno-associated virus (rAAV) particles. For instance, the rAAV particles may be derived from an AAV-producing cell culture. According to the present disclosure, the method can include a) producing a crude cell lysate comprising rAAV particles by hypotonic lysing of the cell culture; b) clarifying the crude cell lysate using filtration producing a supernatant comprising rAAV particles; c) isolating the supernatant to obtain a virus pellet comprising rAAV particles; and d) resuspending the virus pellet with a buffer to obtain purified rAAV particles.

### CROSS REFERENCE TO RELATED APPLICATIONS

This application claims filing benefit of U.S. Provisional Application Ser. No. 63/322,796, having a filing date of Mar. 23, 2022, and U.S. Provisional Application Ser. No. 63/398,622, having a filing date of Aug. 17, 2022, the entire contents of both of which are incorporated herein by reference.

### FEDERAL RESEARCH STATEMENT

This invention was made with government support under 1 R21 AI151475-01A1, awarded by the National Institutes of Health. The government has certain rights in the invention.

### SEQUENCE LISTING

[0002.1] The instant application contains a Sequence Listing which has been submitted electronically in XML format and is hereby incorporated by reference in its entirety. Said XML copy, created on Jun. 1, 2023, is named USC-750_1595_SL.xml and is 2,829 bytes in size.

### BACKGROUND

Adeno-associated virus (AAV) recently has become a popular candidate for use as viral vectors for gene therapy. AAV is non-pathogenic and has an excellent safety profile. AAV is a non-infectious single-strand DNA genome formed by removing most wild-type AAV genome elements, which further reduces the possibility of recombination with any wild-type AAV. Also, AAV has low immunogenicity, with AAV eliciting a mild immune response in vivo. Further, AAV has a broad range of infectivity, wherein AAV can infect both dividing and non-dividing cells in vivo, granting gene delivery to a highly diverse range of cell types. AAVs have stable expression, where AAV does not integrate into the host cell, but rather the genome exists long term as concatemers in non-dividing cells.

Unfortunately, an eight-day-long viral production procedure poses a challenge on keeping the production cost low. AAV is primarily purified using a time-consuming freeze-thaw (FT) lysis method of virus-producing cells. Thus, there exists a need for cost-effective methods to purify AAV particles.

### SUMMARY

The present disclosure is generally directed to a method of purifying recombinant adeno-associated virus (rAAV) particles. For instance, the rAAV particles may be derived from an AAV-producing cell culture. According to the present disclosure, the method includes a) producing a crude cell lysate comprising rAAV particles by hypotonic lysing of the cell culture; b) clarifying the crude cell lysate using filtration, producing a supernatant comprising rAAV particles; c) isolating the supernatant to obtain a virus pellet comprising rAAV particles; and d) resuspending the virus pellet with a buffer to obtain purified rAAV particles.

These and other features and aspects, embodiments and advantages of the present invention will become better understood with reference to the following description and appended claims.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 depicts different stages of water-based hypotonic (WH) lysis of AAV-producing HEK293T cells. 1, 3, and 5 days post-transfected cells were washed and incubated with nuclease-free water supplemented with protease inhibitor.

FIG. 2 depicts a comparison of AAV6 viral titers produced using WHL and FT lysis method.

FIG. 3 depicts Coomassie Brilliant Blue stain of AAV6 samples harvested via freeze-thaw or hypotonic lysis method, 3 days post-transfection. Left two samples are from the first experiment, and right two samples are from the second experiment.

FIG. 4 depicts in vitro transduction efficiency of AAV6 harvested from freeze-thaw lysis or water-based hypotonic lysis.

FIG. 5 depicts mature AAV6 particles under Transmission Electron Microscope in HEK293T cell cytoplasm.

FIG. 6 compares steps of rAAV purification method using hypotonic lysis and FT-lysis method.

Repeat use of reference characters in the present specification and drawings is intended to represent the same or analogous features or elements of the present invention.

### DETAILED DESCRIPTION

Reference will now be made in detail to example embodiments of the disclosure. It is to be understood by one of ordinary skill in the art that the present disclosure is a description of exemplary embodiments only and is not intended as limiting the broader aspects of the present disclosure.

The present disclosure is generally directed to a method of purifying recombinant adeno-associated virus (rAAV) particles. For instance, the rAAV particles may be derived from an AAV-producing cell culture. According to the present disclosure, the method includes a) producing a crude cell lysate comprising rAAV particles by hypotonic lysing of the cell culture; b) clarifying the crude cell lysate using filtration producing a supernatant comprising rAAV particles; c) isolating the supernatant to obtain a virus pellet comprising rAAV particles; and d) resuspending the virus pellet with a buffer to obtain purified rAAV particles.

Advantageously, methods disclosed herein provide a scalable and cost-effective strategy to purify rAAV particles. Contacting rAAV-producing cells with nuclease-free water results in swelling of AAV cells leading to the cells losing their morphology due to passive entry of water molecules into the cells and increased hydrostatic pressure. Thus, the cells become fragile enough to be lysed through gentle shaking or pipetting. Without wishing to be bound by theory, it is understood that the concept of osmosis governs water-based hypotonic lysis. Osmosis is the movement of water molecules from a higher water potential (ψ) region to a lower water potential region through a semi-permeable membrane.

Compared to a solution, distilled water has the maximum concentration of water molecules with the highest possible water potential of zero or ψ=0 MPa. In the presence of solutes, the number of water molecules decreases; hence water potential of a solution is always negative. Therefore, water molecules always move from higher water potential to lower water potentials in solutions with different water potentials separated by a water-permeable membrane. Because of the complex composition of the cell cytoplasm (e.g., Na⁺, K⁺, Ca⁺², metabolites, glucose, amino acids, etc.), the water potential is always negative (ψ distilled water > ψ cell cytoplasm).

According to the present disclosure, the rAAV particles may be derived from AAV-producing cell culture, such as from a cell line which has been adapted for cell culture. The cell line from which the cell culture may be derived includes, but is not limited to, HEK293 cell line, COS cell line, HeLa cell line, BHK cell line, CHO cell line, or Sf9 cell line. For instance, the cell line may be HEK293 cell line. Regardless of the cell line, the cells are inoculated to a culture medium and incubated from about 12 hours to about 30 hours, such as from about 15 hours to about 27 hours, such as from about 18 hours to about 24 hours, or any range therebetween.

In embodiments, the cell culture disclosed herein may be a mammalian cell culture or an insect mammalian cell culture. According to the present disclosure, the cell lysate comprising rAAV particles may be produced using sonication, microfluidization, or lysis methods well known in the art. In one embodiment, the cell lysate may be produced by hypotonic lysis, such as freeze-thaw lysis of cells comprising rAAV particles. In another embodiment, the cell lysate may be produced by water-based hypotonic lysis of cells comprising rAAV particles. Water potential is the measure of water’s potential to do work—the water potential increases or decreases based on the solute concentration in the solution. Water has a lower solute concentration and higher water potential in a hypotonic solution. In a hypertonic solution, water has a higher solute concentration and thus a lower water potential. Distilled water has the highest water potential due to the near absence of solutes. Water passively moves from a higher potential to a lower potential solution; thus, when an animal cell is placed in a hypotonic solution, water will move into the cell and eventually rupture the cell wall via hypotonic cell lysis. Surprisingly, nuclease-free water utilized in methods disclosed herein is able to release rAAV particles via water-based hypotonic lysis from rAAV-producing cells.

According to the present disclosure, following lysis, the cell lysate may comprise a buffer, such as a saline buffer. In one embodiment, the saline buffer includes, but is not limited to, a citrate buffer, a phosphate buffer, an acetate buffer, or a bicarbonate buffer. In one embodiment, the saline buffer comprises a citrate buffer. In another embodiment, the saline buffer comprises a phosphate buffer. In another embodiment, the saline buffer comprises an acetate buffer. In another embodiment, the saline buffer comprises a bicarbonate buffer. The saline buffer may be present in the cell lysate at a concentration from about 5 mM to about 1 M, such as from about 10 mM to about 500 mM, such as from about 50 mM to about 250 mM, such as from about 100 mM to about 200 mM, or any range therebetween.

In another embodiment, the cell lysate further comprises one or more nucleases. For instance, the nuclease may include, but is not limited to, benzonase, lysozyme, β—D—N—acetyl glucosaminidase, Proteinase K, or a combination thereof. The nuclease may be present in the cell lysate at a concentration from about 0.001 mM to about 1 mM, such as from about 0.005 mM to about 0.75 mM, such as from about 0.01 mM to about 0.5 mM, such as from about 0.05 mM to about 0.25 mM, or any range therebetween.

According to the present disclosure, following lysis, the cell lysate may be clarified by filtration, such as microfiltration, ultrafiltration, or nanofiltration, to isolate the rAAV particles. In one embodiment, the cell lysate may be clarified by microfiltration. rAAV particles may be purified and/or isolated from the resulting supernatant using one or more methods well-known in the art for purifying rAAV particles, such as sedimentation, filtration, centrifugation, flocculation, etc. In one embodiment, the rAA V particles may be isolated from the supernatant using centrifugation to pellet the rAAV particles. In one embodiment, the supernatant is centrifuged at from about 15000 rpm to about 20000 rpm, or any range therebetween. In one embodiment, the supernatant may be centrifuged from about 1 hour to about 3 hours, such as 1.5 hours to about 2.5 hours, or any range therebetween. Following centrifugation, the pellet comprising rAAV particles may be dissolved into a saline buffer.

According to the methods disclosed herein, the resuspended pellet comprising rAAV particles may be subjected to one or more additional low speed centrifugation at from about 5000 rpm to about 11000 rpm, such as from about 7500 rpm to about 10000 rpm, or any range therebetween, to remove any remaining impurities thereby purifying rAAV particles.

According to the present disclosure, purified rAAV particles may be further purified by passing rAAV particles through a tangential flow filtration device to remove impurities such as benzonase and/or any low molecular weight protein impurities (e.g., less than about 300 kDa).

According to the present disclosure, the rAAV particles may be a respiratory-specific rAAV serotype. In one embodiment, the serotype of rAAV may include, but is not limited to, rAAV1, rAAV2, rAAV3, rAAV4, rAAV5, rAAV6, rAAV7, rAAV8, rAAV9, rAAV10, rAAV11, rAAV12, rAAV13, or other rAAVs with engineered capsids to achieve tissue and cell specificity. In one embodiment, the rAAV particle may be rAAV2, rAAV6, rAAV8, or rAAV9. In one embodiment, the rAAV particle may be rAAV6. In

According to the present disclosure, the purity and/or quantity of rAA V particles may be measured using an immunoassay, a nucleic acid hybridization-based assay (e.g., a dot-blot assay), SDS-PAGE followed by Coomassie Brilliant Blue dye staining, visualization with an electron microscope, a PCR assay, a green fluorescent cell assay, or combinations thereof. For instance, the purity and/or quantity of rAAV particles may be measured using SDS-PAGE followed by Coomassie Brilliant Blue dye staining to detect the major rAAV capsid proteins VP1, VP2, and VP3. It is understood that the quantity of VP1, VP2, and VP3 detected are indicative of the amount of rAAV particles present in the preparation. In one embodiment, the rAAV particles purified according to methods disclosed herein may be more than about 80% pure, such as more than 85% pure, such as more than 90% pure, such as more than 95% pure, such as more than 97% pure, such as more than 98% pure, such as more than 99% pure.

According to the present disclosure, the rAAV particles may be purified and/or concentrated using centrifugation, tangential flow filtration, diafiltration, or a combination thereof.

In one embodiment, the rAAV particles comprise an adeno-associated viral vector. “Viral vector” as used herein refers to a recombinant adeno-associated virus that is capable of delivering an exogenous nucleotide sequence into a host cell. The viral vector may be prepared by a polymerase chain reaction (PCR) amplification of the targeted nucleotide sequence encoding a polypeptide or heterologous gene of interest. In one embodiment, the heterologous gene may be a sequence encoding a protein or nucleic acid which may be used for gene therapy. In one embodiment, the heterologous gene encodes a nucleic acid, such as a ribonucleic acid (e.g., a short interfering RNA (siRNA), double stranded RNA (dsRNA), micro RNA (miRNA), a short hairpin RNA (shRNA), etc.). In one embodiment, the heterologous gene may be a sequence encoding a protein. For instance, the encoded protein may be a Spike protein (S protein), such as SARS-CoV-2 S protein.

Methods disclosed herein may yield purified rAAV particles comprising a viral titer of from about 5.0 x 10¹³ viral genome (vg)/mL to about 3.0 x 10¹⁵ vg/mL, such as about 1.0 x 10¹⁴ to about 2.0 x 10¹⁵, or any range therebetween.

Methods disclosed herein yield AAV particles purified via water-based hypotonic lysis that are comparable in terms of viral titer, sample purity, or in transduction efficiency compared to AAV particles purified via freeze-thaw lysis methods. These implications support the water-based hypotonic lysis method as a suitable, if not superior, method of cell lysis compared to freeze-thaw lysis in the harvesting of Adeno-Associated Virus. In the event of skipping the step of using salts (e.g., ammonium sulfate), glycols (e.g., polyethylene glycol), or solvents (e.g., methanol) to precipitate out virus lost in cell supernatant, one may proceed with low-speed centrifugation, sterile filtration, and/or highspeed centrifugation to purify, isolate, and concentrate AAV. This can significantly cut down on the amount of time required to produce AAV from a week or more to less than 5 days, e.g., 3 or 4 days, and in doing so can reduce the cost and difficulty of producing this otherwise expensive gene vector.

With AAV becoming a key go-to gene vector in current clinical research, future research will continue to rely on it due to its ability to efficient and predictable incorporation of genetic material into host cells with minimal immune reaction or host response. Encouraging the adoption of such competitive procedures will be highly beneficial in the field of molecular biology in hastening the production of AAV.

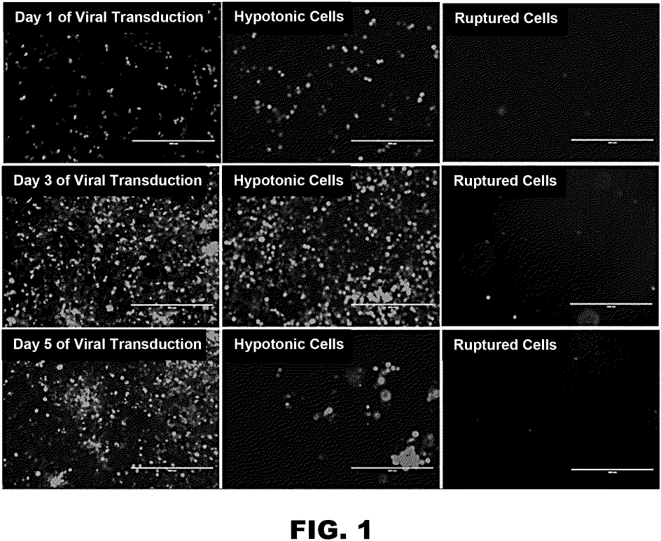

FIG. 1 depicts the efficiency of water-based hypotonic lysis in the destruction of host cells where the swelling and pipetting of cells resulted in a near-complete lysing of cells present. This is an interesting finding considering the various defenses cells have in maintaining an isotonic state, yet a hypotonic state was reached in just 10 minutes. In the freeze-thaw method, virus inevitably gets partially stuck in the cell debris, resulting in a loss of product. Comparison of product loss in the freeze-thaw vs. the water-based hypotonic lysis method can be determined through known methods, e.g., transmission electron microscopy or by Coomassie Brilliant Blue staining to visualize the presence of VP1, VP2, and VP3 protein.

Surprisingly, the usage of pure water to induce a hypotonic state in HEK293T embryonic kidney cells allows for efficient lysing of cells. Methods disclosed herein allow for near complete cell destruction allowing for AAV particles to be released and collected in solution. Interestingly, the transduction efficiency, final viral titer, and purity of AAV produced via water-based hypotonic lysis is on par with AAV produced from the freeze thaw method. The implications of this finding may allow for AAV to be produced significantly faster than relying on freeze thaw lysis, as the virus can in some embodiments be processed the same day. This may aid in research by essentially saving a day of processing by circumventing three cycles of 2 hours at -80° C. followed by 20 minutes at 37° C.

A viral vector comprising shRNA targeting SARS-CoV-2 spike protein may be produced utilizing methods disclosed herein, which could provide a fast method of producing antiviral treatments for COVID-19 infection. This could significantly aid in better controlling the pandemic by inventing a new therapeutic capable of inhibiting the spread of infectious virions by performing spike protein knockout in vivo. These implications may be quickly implemented, as the testing guidelines for developing COVID-19 antivirals has been significantly hastened due to the severity of the global pandemic.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Methods

### Suppliers and Materials

Cells: Human embryonic kidney cells (HEK293T) obtained from the University of South Carolina VVC; Human liver Huh7.5 cell (University of North Carolina); Monkey kidney Vero cell, (Penn State University) and Monkey kidney Cos-1 cell, (University of South Carolina). Each cell type was grown in Dulbecco’s Modified Eagle Medium (DMEM) supplemented with 10% fetal calf serum (FCS; Invitrogen #S11950), 1X penicillin and streptomycin (Sigma #P4333-100 mL), and 2 mM L-glutamine (Invitrogen 02956-100).

Plasmids: pXR6, pHelper, and pLuc-shRNA plasmids were acquired from Viral Vector Core Facility School of Medicine, University of South Carolina.

Chemicals: MEM powder and Sodium Bicarbonate and L-glutamine were purchased from Sigma, Penicillin and Streptomycin (100X) purchased from Gibco. SDS-PAGE reagents and Coomassie Brilliant Blue G-250 dye (Cat #1610437) were purchased from BioRad. Polyethyleneimine hydrochloride (PEI) was purchased from Sigma (Cat #764647).

### AAV Production and Purification

Each 6 well culture plate coated with 0.2% gelatin was seeded with 1x10⁶ HEK293T cells in 2 mL of MEMG with 10% of FCS. Cells were incubated for 24 hours at 37° C. in 5% CO₂ incubator. The AAV cis (pXR6), AAV trans, and pHelper plasmids (3.3 µg of each) and 3.3 µg of AAV plasmid with gene of interest were added to 200 µl of sterile 150 mM NaCl solution. Polyethylenimine stock solution was prepared at 20 mg/ml in sterile water, and the pH was adjusted to 4.5 with sodium hydroxide. Also, 1.5 µL of PEI stock was added to a new tube with 200 µl of plasmid solution and mixed by vortexing. After 10 minutes of incubation at room temperature, the plasmid and PEI solutions were mixed and incubated for another 10 minutes. In the meantime, the media was replaced with 5% MEMG (450 mL MEM, 50 mL CS, 5 mL 100x PLS, and 5 mL of Glutamine) without antibiotics. After the incubation period, 400 µl of PEI-DNA mixture was added dropwise to the 6 well culture plate. Cultures were incubated at 37° C. in 5% CO₂ incubator for 24 hours. On the next day, media was replaced with 2 mL of virus-producing medium 5% MEMG containing 2 mM L-glutamine, and 1 mM sodium pyruvate, and 20% glucose (450 mL MEM, 25 mL CS, 10 mL of Sodium bicarbonate, 5 mL Sodium pyruvate, and 5 mL 20% glucose). Subsequently, 24 h, 72 h, and 120 h post-transfection, the culture supernatants were harvested and centrifuged at 3000 rpm for 5 minutes to remove cell debris. Polyethylene glycol was added in a 1:4 ratio and incubated overnight to precipitate any viral particles in the culture supernatant while cells were collected and lysed using a lysis method described herein.

### Water Based Hypotonic Lysis Method

Following the desired incubation, cells were washed twice with nuclease-free water, and 1 mL of nuclease-free water supplemented with 1x protease inhibitor mixture cocktail was added. For hypotonic cell swelling, cells were incubated at 37° C. for 10 minutes. Following incubation, cells were pipetted rigorously 10 times to induce lysis (FIG. 1). Then, 100 µl of 10x PBS were added promptly to each well. The lysates were transferred to 1.5 mL microcentrifuge tubes, passed through 0.45 µM syringe filters, and processed for virus purification as described herein.

### Freeze-Thaw (FT) Hypotonic Lysis Method

The cell monolayers were scrapped, suspended in 1 mL PBS supplemented with 30 µl 5 M NaCl and 10 µl protease inhibitor cocktail, and transferred to a 1.5 mL microcentrifuge tube. The samples were freeze-thawed thrice with at least 3 hrs of freezing time and 15 minutes thawing. Following the third freeze-thaw, cells were sonicated for 15 seconds to lyse the remaining unlysed cells and shear genomic DNA. 1 µL of 100 mM benzonase (to degrade DNA and RNA) was added and incubated in a 37° C. hot water bath for 2 hours. Freeze-thaw samples and the PEG precipitated virus culture supernatants were centrifuged at 6000 rpm for 10 minutes. Supernatant from FT-lysis and the pellet from the PEG precipitate were mixed and passed through a 0.45 µM syringe filter to remove any cellular debris and further processed for virus purification.

### Virus Purification

The virus supernatants reinforced through a 0.45 µM filter were centrifuged at 19,000 rpm for 2 hours at 4° C. to pellet the virus particles. The virus pellet was dissolved in 200 µl of virus suspension media. The virus suspension was transferred to a new 1.5 mL microcentrifuge tube and centrifuged twice at 10000 rpm for 1 minute to pellet out the remaining impurities. Finally, the viral supernatant was aliquoted in sterile 0.2 mL microcentrifuge tubes and stored at -80° C. deep freezer until use.

### AAV Titration by Real-time RT-PCR

A standardized real-time PCR used at the viral vector core facility was used to quantitate the AAV preparations. In brief, commercially obtained AAV standard was serially diluted to 2x10⁵, 2x10⁶, and 2x10⁷ genomic copies. The Q-PCR was performed using SYBR Green PCR Master mix (Fisher Scientific #4309155) and MyiQ Bio-Rad single color real-time PCR detection system. The ITR forward primer (5′ GGAACCCCTAGTGATGGAGTT 3′ (SEQ ID NO: 1)) and the ITR reverse primer (5′ CGGCCTCAGTGAGCGA 3′ (SEQ ID NO: 2)) were added to the reaction with the final concentration of 300 nM in 20 µL of total volume. 5 µL of sample or standard are added to 20 µL of primers and SYBR mixture. The nucleic acid amplification was performed under the following PCR conditions: 95° C. for 10 minutes, 95° C. for 15 s, 60° C. for 60 s, for 40 cycles. The cycle values were entered into a data sheet, and viral titers were calculated and presented as genome copies/ml.

### Coomassie Brilliant Blue Staining

Coomassie Brilliant Blue staining of SDS PAGE was performed to determine the purity of the rAAV produced by FT-lysis and WH-lysis method. 5 µL of 1x10¹² GC of each virus sample mixed with 4% mercaptoethanol was added to NuPage sample buffer and boiled at 90° C. for 5 minutes. Samples were separated on 7% SDS PAGE followed by Coomassie Brilliant Blue staining. Excess dyes were washed with destain solution twice in DI water for 10 minutes before being scanned in an LI-COR Odyssey Infrared Scanner.

### Transduction Efficiency

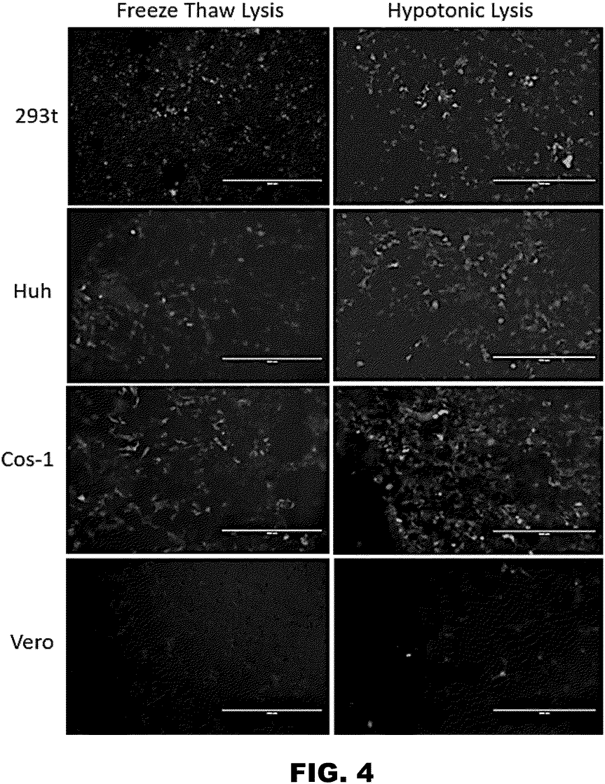

The GFP expression was evaluated to compare the transduction efficiency of the AAV6 produced by the FT-lysis and WH-lysis methods. 3x10⁴ cells of human and monkey origin, as listed in FIG. 4, were seeded in a gelatin-coated 96 well plate. The next day media was removed, and cells were infected with 30 µL of 1x10¹² GC of virus preparation diluted in MEMG without FCS. Cells were incubated at 37° C. for 2 hours, and the virus was removed, and 100 µL of complete media was added to each well. Forty-eight hours post-transfection, cells were imaged with AMG EVOS fluorescence microscope.

### Transmission Electron Microscopy (TEM) Preparation

For TEM, 120 hours post-transfection with AAV cis and trans plasmids, cells were fixed in 3% glutaraldehyde in PBS for 1 hr at room temperature, washed with 0.1 M cacodylate buffer, and incubated in 1% OsO4 (in 0.1 M cacodylate buffer) for 40 min at room temperature. Samples were then washed 5 times with distilled water. Fixed cells were scraped, transferred to a micro centrifuge tube, and pelleted at 4000 rpm for 5 minutes. Then, 500 µL 2% warm agar (approximately 50° C.) was added to each pellet, with cell clumps being mixed with a plastic toothpick. These tubes were then centrifuged at 12,000 rpm for 5 minutes to concentrate cells into a pellet. Samples were kept at 4° C. for 15 minutes to solidify agar. The cell pellet was then sliced into rectangular pieces of 2 mm to 3 mm size and stored at 4° C. overnight in DI water. The following day, samples were dehydrated through a series of solvents: 2 x 15 minutes (70%, 95%, 100% ethanol), 1:1 ratio of ethanol and acetonitrile for 10 minutes, 2 x 15 minutes of 100% acetonitrile, then 1:1 ratio of acetonitrile to PolyBed-812 for 1 hour. Then the samples were placed into rubber molds with PolyBed-812 and moved to a 60° C. oven for 36 hours to allow for the resin to harden. Resin blocks were then trimmed using a glass razor blade and a diamond sectioning knife, from which 200 nm sections were obtained and mounted on EM copper grids with carbon coating. Sections were post-stained in 2% uranyl acetate in water and Reynolds’ lead citrate for 10 min each and then processed for TEM imaging using JEM-1400 Plus Electron Microscope.

### Example 1

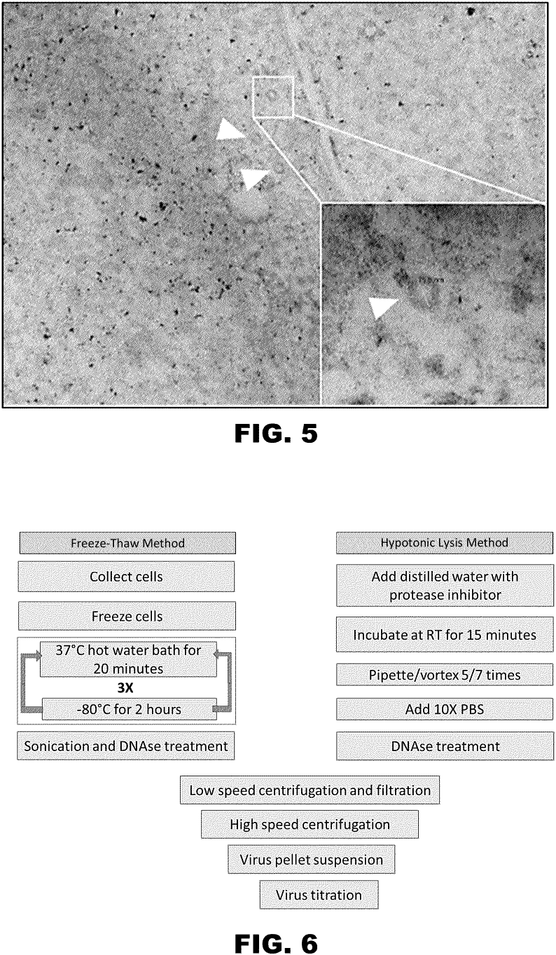

AAV titration: To evaluate and compare the virus harvesting efficiency of the conventional FT-lysis method with our novel WH-lysis method of AAV purification, three independent experiments were performed, and the viral titers were compared. AAV6, a respiratory-specific AAV serotype expressing luciferase shRNA under the U6-promoter and GFP marker under CMV-promoter, was selected for this experiment. AAV6 expressing SARS-CoV-2 S-protein specific shRNA were also produced. The number of rAAV6 genome copies (GC) and the titer for individual preparations were determined by SYBR green-based quantitative PCR targeting the ITR region of the AAV genome and presented in FIG. 2. Virus harvested on 1-, 3-, and 5-days post-transfection were purified and titrated and the results are presented in FIG. 5. The virus yield was optimal and greater than 1E + 13 genome copies per mL (GC/mL) on day 3. This titer is comparable to most commercially available rAAV stock.

As noted above, AAV6 expressing SARS-CoV-2 shRNA was prepared, and its titer are listed in Table 1. SYBR green-based quantitative PCR targeting the ITR region of the AAV genome (AAV6 luciferase shRNA) is presented in FIG. 2. Virus harvested on 1-, 3-, and 5-days post-transfection were purified and titrated and the results are presented in FIG. 5. The virus yield was optimal and greater than 1E + 13 genome copies per mL (GC/mL) on day 3. This titer is comparable to most commercially available rAAV stock. AAV6 expressing SARS-CoV-2 shRNA was also prepared, and its titer are listed in Table 1.

TABLE 1

Viral titers of AAV6 samples with incorporated shRNA or Luciferase plasmids

| | |
|---|---|
| Plasmid Candidate | Titer (Viral genomes/mL) |
| AAV-6 Luciferase | 9.18 × 10¹³ |
| AAV6-106 | 6.55 × 10¹³ |
| AAV6-264 | 2.84 × 10¹⁴ |
| AAV6-299 | 2.54 × 10¹⁵ |
| AAV6-413 | 2.47 × 10¹⁵ |

### Example 2

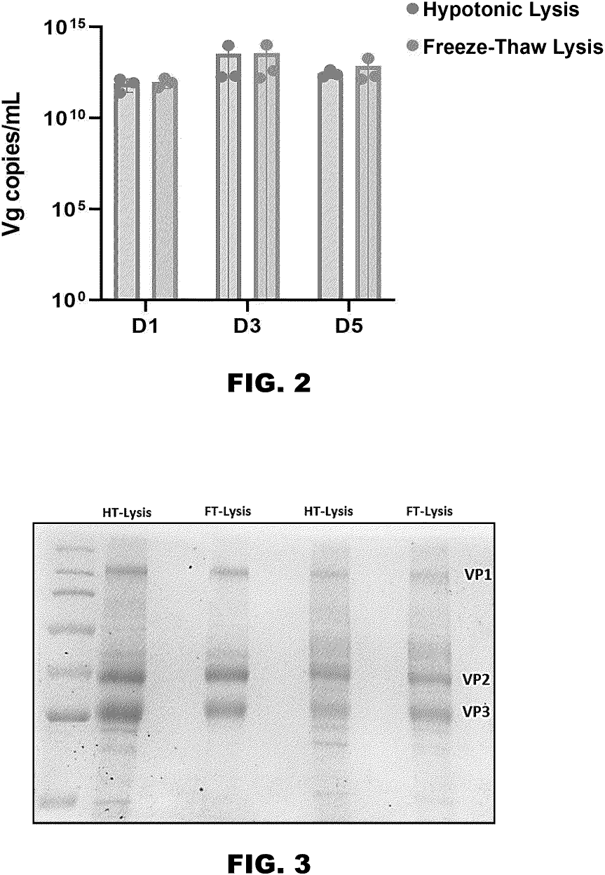

Virus purity: To test the purity of viral preparations, the same amount of protein samples were resolved via SDS-PAGE and stained for protein content with Coomassie Brilliant Blue dye (FIG. 3). The major rAAV capsid proteins VP1, VP2, and VP3 were detected using this staining method. The amount of VP1, VP2, and VP3 are indicative of the amount of rAAV particles present in the preparation. The result indicates that the purity of AAV prepared by the WH-lysis method is comparable to the FH-lysis method.

### Example 3

Transduction efficiency: The transduction efficiency or the gene delivery ability is the ultimate assessment of the quality of the virus prepared by WH-lysis method. In two-chambered slides seeded at a density of 1x 10⁵ cells of HEK293T, Huh7.5, Cos-1, and Vero cells are infected with 1x10¹² GC of the rAAV6 virus. On day 3 post-transduction, the cells were analyzed for GFP expression. The results demonstrate that rAAV6 could transduce all cell lines except Vero and the GFP expression pattern revealed that the WH-lysis method’s transduction efficiency was better than the FT-lysis rAAV purification method.

### Example 4

TEM analysis of the rAAV6: To investigate the assembly of rAAV, HEK293T cells were supplied with necessary components from AAV and adenovirus that are encoded in three plasmids. pHelper plasmid encodes for E4, E2a, and VA from Adenovirus, pXR plasmids are serotype specific and provide Rep and Cap. Transfer plasmid provides the transgene (shRNA sequence or an open reading frame) placed between the two ITRs.

TEM analysis of HEK293T cells transfected with these plasmids revealed several hollow electron-dense of HEK293T cells transfected with these plasmids revealed several hollow electron-dense structures in the cytoplasm representing assembled and packaged rAAV. These structures were measured to be 25 nm in diameter matched the expected size of AAV particles. Real-time positivity of these cell lysates with AAV-specific primer sets supported these structures as rAAVs.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method of purifying recombinant adeno associated virus (rAAV) particles from an AAV-producing cell culture, the method comprising:
hypotonic lysing of the cell culture to produce a crude cell lysate comprising rAAV particles by;
filtering the crude cell lysate and thereby clarifying the crude cell lysate to produce a supernatant comprising rAAV particles;
isolating the supernatant to obtain a virus pellet comprising rAAV particles; and
resuspending the virus pellet with a buffer to obtain purified rAAV particles.

2. The method of claim 1, wherein the rAAV particles comprise rAAV1, rAAV2, rAAV3, rAAV4, rAAV5, rAAV6, rAAV7, rAAV8, rAAV9, rAAV10, rAAV11, rAAV12, or rAAV13.

3. The method of claim 1, wherein the rAAV particles comprise rAAV2, rAAV6, rAAV8, or rAAV9.

4. The method of claim 1, wherein the rAAV encodes an shRNA.

5. The method of claim 1, wherein the rAAV encodes an shRNA for a Spike protein (S protein).

6. The method of claim 1, wherein the rAAV encodes an shRNA for SARS-CoV-2 S protein.

7. The method of claim 1, wherein the step of hypotonic lysis comprises water-based hypotonic lysis.

8. The method of claim 7, wherein the water-based hypotonic lysis utilizes nuclease-free water.

9. The method of claim 1, wherein the supernatant is isolated by use of centrifugation.

10. The method of claim 9, wherein the supernatant is centrifuged at from about 15000 rpm to about 20000 rpm.

11. The method of claim 1, wherein the cell culture comprises a mammalian cell culture or an insect cell culture.

12. The method of claim 1, further comprising centrifuging the resuspended virus pellet at from about 5000 rpm to about 11000 rpm.

13. The method of claim 1, wherein the supernatant comprises one or more nucleases.

14. The method of claim 13, wherein the one or more nucleases comprises Benzonase, lysozyme, β—D—N—acetyl glucosaminidase, Proteinase K, or a combination thereof.

15. The method of claim 1, further comprising passing the rAAV particles through a tangential flow filtration.

16. The method of claim 1, wherein the purified rAAV particles comprise a viral titer of from about 5.0 x 10¹³ vg/mL to about 3.0 x 10¹⁵ vg/mL.
